(19) United States
(12) Patent Application Publication
(10) Pub. No.: US 2025/0064904 A1
(43) Pub. Date: Feb. 27, 2025

(54) METHOD FOR PREVENTING HUMAN GLANDS FROM RADIATION DAMAGE

(71) Applicant: MERZ THERAPEUTICS GMBH, FRANKFURT AM MAIN (DE)

(72) Inventors: JOERG MUELLER, BERLIN (DE); RICHARD BAUM, HARGESHEIM (DE)

(73) Assignee: MERZ THERAPEUTICS GMBH, Frankfurt am Main (DE)

(21) Appl. No.: 17/767,725

(22) PCT Filed: Oct. 12, 2020
(86) PCT No.: PCT/EP2020/078619

§ 371 Date: Apr. 8, 2022

(30) Foreign Application Priority Data
Oct. 10, 2019 (DE) 10 2019 215 585.5

(51) Int. Cl. A61K 38/48; A61K 31/46; A61P 39/00

(52) U.S. Cl. CPC A61K 38/4893; A61K 31/46; A61P 39/00

(57) ABSTRACT

The invention relates to the field of radiation oncology. The method according to the invention comprises the use of botulinum toxin and an anticholinergic to prevent glands from radiation damage and side-effects caused by to radioligands. The method according to the invention further comprises the use of botulinum toxin to prevent glands from radiation damage, wherein radiation damage is caused by radioligands. By using the method according to the invention, the present invention can provide effective protection of the glands in order to avoid radiation damage.

### CROSS-REFERENCE TO RELATED APPLICATIONS

This application is a national phase entry under 35 U.S.C. § 371 of International Patent Application PCT/EP2020/079806, filed Oct. 12, 2020, published as International Patent Publication WO 2021/069740 on Apr. 15, 2021, which claims the benefit of German Patent Application DE 10 2019 215 585.5, filed on Oct. 10, 2019, the contents of all are hereby incorporated by reference.

### FIELD OF INVENTION

The present invention relates to the field of radiation oncology. Using botulinum toxin and anticholinergic can avoid radiation damage by radioligands in human glands. An optimized therapy regimen can impart further advantageous properties to the method. This includes both diagnostic imaging procedures and therapeutic procedures.

### BACKGROUND OF THE INVENTION

A radioligand is a substance labeled with a radionuclide, which can bind as a ligand to a target protein, such as, for example, a receptor. Such radioligands are used, for example, both in the diagnosis and therapy of oncological diseases. A prominent example is PSMA radionuclides in the diagnosis and treatment of prostate carcinoma.

PSMA (prostate-specific membrane protein) is a transmembrane protein that is expressed by prostate carcinoma cells up to 1,000 times more than by normal prostate cells. PSMA is therefore an ideal target protein for the diagnosis and at the same time for the targeted therapy of prostate cancer. PSMA is also expressed by other carcinomas, although at lower levels compared to prostate carcinoma. PSMA is also expressed by salivary glands and ocular glands. Permanent damage to the glands mentioned leads to severe dry mouth with a variety of complications, such as dental damage and chronic dry eyes, which can lead to visual impairment.

PSMA radionuclides are used both diagnostically and therapeutically.

The PSMA radioligands 68Ga-PSMA and 18F-PSMA are used to diagnose primary and recurrent prostate cancer and to search for metastases. The diagnostic ligands accumulate in the prostate carcinoma cells and the other organs/tumors mentioned above, and the diagnostic PSMA radioligands can damage ocular and salivary glands.

The PSMA radioligands used therapeutically always cause damage to the salivary glands, which varies in severity and often persists depending on the radioligand, with significant health problems and reduced quality of life (dry mouth, ulcers in the entire mouth area, dysphagia, risk of aspiration, dental damage, etc.) as a result. Another related problem is that the PSMA radioligands are often administered repeatedly, such that the gland damage accumulates. PSMA radioligands also cause ocular gland damage, which varies in severity depending on the radioligand, with health implications: dry eyes, repeated ocular inflammation, chronic ocular inflammation and visual impairment. This damage caused by PSMA therapy is often irreversible. Furthermore, this damage and complications often mean that prostate cancer patients are no longer able to take advantage of the life-prolonging therapy.

Presumably, different PSMA radioligands have different affinity for the individual salivary glands, and thus different toxicity. The various radionuclides are either alpha, beta or gamma emitters. For example, the nuclides 177Lu (a beta emitter with a range of 2 mm) and 225Ac (an alpha emitter with a range of 50-90 μm) are currently being used. In the future, even more potent PSMA radioligands that radiate strongly over a very short distance (50-90 μm) are to be expected for therapeutic use. There is growing evidence that ²²⁵Ac damages the salivary glands more than does ¹⁷⁷Lu.

With the use of even more potent radioligands, such as alpha emitters, glands are at an even greater risk of being damaged.

Glands perform a variety of tasks in the human body. A distinction is made, among other things, between endocrine and exocrine glands. If a target is not only expressed in a tumor, for example, but also in glands, such as PSMA expression in salivary glands and also ocular glands, healthy glands will also be attacked.

This is where botulinum toxin can help: Botulinum toxin injections can protect the glands as part of a coordinated procedure. Botulinum toxin is a generic term for the family of toxins produced by the bacterium Clostridium botulinum. There are currently seven different known botulinum toxins (A, B, C, D, E, F and G) that differ in their properties. The mechanisms of action of the various botulinum toxin types also differ: for example, the main effect of type A is based on the cleavage of intracellular SNAP-25, while type B mainly cleaves the “vehicle associated membrane protein” (=VAMP). Botulinum toxin type B also has other differences compared to type A: Type B binds presynaptically to the Syt II protein, there is also partial presynaptic interaction between type B and tetanus toxin residues and neurotoxin-ganglioside interaction with the gangliosides GD1a, GD1b and GM1a. Local botulinum toxin injections lead to a dose-dependent decrease in saliva production after a few days, so the onset of action is a few days. Effectiveness, duration of action and side effects are mainly dependent on the dose to be administered. In general, the duration of action is between four and 16 weeks, with the duration of action depending on the type used: types E and F have a significantly shorter duration of action of around three to six weeks. Botulinum toxin does not permanently or irreversibly damage the salivary glands. The salivary gland function is completely restored under botulinum toxin over time. The injections can be repeated as often as desired without organ damage.

The mechanism of action of botulinum toxin on the salivary glands is known and has been described in detail previously. All botulinum toxin preparations and types (A-G) induce a dose-dependent denervation of the glands 1-10 days, i.e., variable in time, after injection and thus a temporary dose-dependent decrease in salivary secretion. Type B has a comparatively quick onset of action of around three days, while Type A has an onset of action of around six days. The decrease in salivary secretion affects both serous and mucous gland parts of the relevant salivary glands. The decrease in gland function reduces saliva production and the blood flow to the glands. Both mechanisms (individually or combined) result in a reduced uptake of the harmful radionuclides due to reduced glandular activity during radionuclide-PSMA therapy in the glands and permanent such that irreversible damage to the glands is prevented. Here, too, there is a difference between botulinum toxin type B and type A: Type B has a higher affinity for autonomic nerve endings than type A. This is of particular importance since the salivary glands (in contrast to the muscles) are exclusively autonomously innervated. Therefore, botulinum toxin type B has a stronger and longer-lasting effect on the salivary glands than type A. Compared to type A, local injection with type B leads to a greater spread in the body with a higher affinity to autonomic structures. Accordingly, when distant glands are injected, Type B can result in autonomic effects on uninjected glands, such as the ocular glands. This results in a certain protective effect on the ocular glands through botulinum toxin type B injection into the salivary glands. This means that a salivary gland injection with botulinum toxin type B can have a certain protective function due to a higher affinity to autonomous structures and a greater regional spread to the glands, which are otherwise not directly accessible to botulinum toxin injections, such as the sublingual salivary gland, minor salivary glands, and ocular glands. This mechanism of action applies to all glands treated with BTX, even if the degree of denervation with reduction in saliva production may vary in the individual glands.

Salivary glands also have androgen receptors. The majority of patients with advanced prostate carcinoma receive antiandrogenic therapy. Antiandrogens markedly increase PSMA uptake, resulting in potentiation of salivary gland damage by PSMA radioligands. Therefore, the present invention is particularly suitable for patients who have already undergone antiandrogenic therapy or are undergoing radioligand therapy, since they are particularly susceptible to glandular damage by PSMA radioligands.

Also, all salivary glands express numerous α-1 adrenoreceptors. The stimulation of α-1 receptors leads to significant salivary gland activation and salivary secretion. Botulinum toxin type A results in down-regulation of the α-1 adrenoreceptors and also in reduced salivary gland activation and reduced PSMA radioligand binding to the salivary glands injected with botulinum toxin.

Furthermore, aquaporins lead to significantly increased PSMA radioligand binding, particularly in the submandibular gland. Aquaporins are proteins that form channels in the cell membrane in order to facilitate the passage of water and other molecules, such as metalloids. The radionuclides are predominantly metalloids. In particular, the submandibular gland expresses high levels of aquaporins (AQP) in the apical membrane of the acinar cells of the salivary glands and particularly the submandibular gland. After about one to two weeks, botulinum toxin type A and type B significantly reduce aquaporin mRNA (e.g., AQP 5) in the submandibular gland and the AQP distribution in the apical membrane. In addition, botulinum toxin type A and type B induces acini apoptosis. Thus, botulinum toxin injections into the submandibular gland result in a significant decrease in aquaporin-mediated radionuclide uptake into the cell and thus protect the salivary glands, particularly the submandibular gland, from permanent damage. When the botulinum toxin effect subsides, the aquaporin changes mentioned recede completely.

In particular, the frequently required repetition of radionuclide PSMA therapy (usually up to four treatments within six months) leads to cumulative permanent massive damage to the salivary glands. The botulinum toxin effect subsides after about six to sixteen weeks without any consequences and the salivary gland function starts again.

Not only botulinum toxin, but also anticholinergics can protect the salivary and ocular glands. Anticholinergic agents, also called parasympatholytics, suppress the effects of acetylcholine by competitively inhibiting the acetylcholine receptor. This blocks the nerve stimuli that lead to the increased secretion of the glands. Thus, the protective effects of botulinum toxin and anticholinergics complement and potentiate each other: botulinum toxin inhibits the release of acetylcholine into the synaptic cleft (presynaptically) and the anticholinergics block the acetylcholine receptors (postsynaptically). Only the combined pre- and postsynaptic blockade by either substances (botulinum toxin and anticholinergics) enables the optimal protective effect on the salivary and ocular glands during radioligand therapy. During radioligand therapy or the use of radioligands for diagnostics, the parasympathetic fibers increase saliva production and serous saliva is primarily produced in the parotid gland, which is why this function is particularly protected by the systemically effective anticholinergics during PSMA radionuclide therapy. This effect is utilized in the present invention as a protective effect of the glands. Anticholinergic agents are dose-dependently and systemically effective and can therefore reach glands that are not directly treatable with botulinum toxin injection, including the sublingual gland, the minor salivary glands, and the ocular glands. Due to their anatomical location and the risk of side effects, these glands are not directly treatable with botulinum toxin. Thus, these glands, such as the ocular glands, are protected by anticholinergics, such that they are not permanently damaged by a radioligand and thus dehydration of the eyes and chronic severe inflammation of the eyes and lid margins, which severely restricts the quality of life, are avoided. In addition, anticholinergics enhance the botulinum toxin effect on, for example, the parotid and submandibular glands.

In particular, the submandibular gland benefits from the combination treatment of botulinum toxin and anticholinergic, since this gland is seromucous. The double protection ensures that the patient can prepare the food mechanically in the oral cavity during the chewing process, such that the food pulp continues to slide smoothly and digestion is not impaired. Furthermore, saliva contains antimicrobial components and thus ensures the remineralization of the teeth, such that dental damage is avoided by the double protection.

The protective effect on the glands can be further enhanced by a combination of botulinum toxin and anticholinergic. The combination with an anticholinergic leads to an enhancement of the botulinum toxin effect on the parotid gland and submandibular gland and to a partial effect at the other salivary glands and ocular glands for the duration of the effectiveness of the respective anticholinergic. Therefore, a combination of botulinum toxin and anticholinergic is most effective in terms of salivary gland secretion, followed by botulinum toxin monotherapy, followed by anticholinergic monotherapy.

However, not all patients may benefit from a combination treatment consisting of botulinum toxin and anticholinergic: Patients with cognitive disorder, including all severities of a cognitive disorder (from mild to moderate to severe cognitive disorder) should not take an anticholinergic, as cognition-altering effects can occur. In other words, there is a contraindication. Likewise, patients with cardiac arrhythmias should not be treated with anticholinergics, because anticholinergics increase the heart rate and conduction disorders, tachyarrhythmias, heart failure and angina pectoris attacks may also occur. Here too, there is a contraindication. Therefore, these patient groups exposed to radioligand diagnostics or therapy are treated with botulinum toxin without an anticholinergic. Here, botulinum toxin A exerts its effect both directly in the injected gland and by passive diffusion in adjacent glands. For example, if botulinum toxin type A is injected in the submandibular gland on both sides, the sublingual gland is also protected by diffusion. Botulinum toxin B exerts its effect both directly in the injected gland and by passive diffusion in other glands, with the diffusion radius being greater than that of botulinum toxin type A. In addition, botulinum toxin type B has a higher affinity for autonomic structures, resulting in botulinum toxin type B also acting on distant glands that were not injected with botulinum toxin type B, such as the ocular glands, sublingual gland, and minor salivary glands.

In summary, there is an urgent need for a method for protecting in particular the vital ocular and salivary glands, which are not the target structure of radioligand diagnostics or radioligand therapy in the context of diagnostics or therapy.

The present method of the invention offers a preventative solution to prevent irreversible gland damage by diagnostic or therapeutic radioligands.

### SUMMARY OF THE INVENTION

The present invention relates to botulinum toxin in combination with an anticholinergic for use in a method of preventing radiation damage to the glands, wherein radiation damage is caused by radioligands. Furthermore, the present invention also encompasses botulinum toxin for use in a method of preventing radiation damage to the glands, wherein the radiation damage is caused by radioligands.

In some embodiments, botulinum toxin is used in combination with an anticholinergic for use in a method, wherein the radiation damage is caused by PSMA radioligands

In some embodiments, botulinum toxin is used in combination with an anticholinergic for use in a method, wherein the radiation damage is caused by ²²⁵Ac-PSMA-617, ⁶⁸Ga-PSMA, ¹⁸F-PSMA or ¹⁷⁷Lu-PSMA.

In some embodiments, the botulinum toxin is selected from the group consisting of botulinum toxin types A, B, C, D, E, F and G.

In some embodiments, the botulinum toxin is a botulinum toxin type A.

In some embodiments, the botulinum toxin comprises between 1 and 10,000 units.

In some embodiments, the botulinum toxin comprises between 1 unit and 1,500 units of type A.

In some embodiments, the botulinum toxin is a botulinum toxin type B.

In some embodiments, the botulinum toxin comprises between 100 and 10,000 type B units.

In some embodiments, the botulinum toxin is a botulinum toxin type E or type F.

In some embodiments, the botulinum toxin comprises 1 and 10,000 units of type E or type F.

In some embodiments, the anticholinergic is selected from the group consisting of tropicamide, atropine, scopolamine, glycopyrrolate, amitriptyline, clonidine, ipratropium bromide, and trihexyphenidyl. Also encompassed are all other pharmaceuticals that act as an anticholinergic or parasympatholytic agent.

In some embodiments, the anticholinergic is to be administered transdermally, orally, or intravenously.

In some embodiments, the anticholinergic is scopolamine, wherein the scopolamine is to be administered transdermally.

In some embodiments, the botulinum toxin is botulinum toxin type B for the prevention of radiation damage to the parotid, submandibular, sublingual, minor salivary and ocular glands, wherein radiation damage is caused by radioligands.

In some embodiments, the botulinum toxin is botulinum toxin type A for lowering aquaporin-mediated radionuclide uptake in the submandibular and salivary gland cells.

In some embodiments, the botulinum toxin is botulinum toxin type A, wherein the botulinum toxin type A is to be administered in a dose ratio of 2/3 into the parotid glands and 1/3 into the submandibular glands.

In some embodiments, the botulinum toxin is botulinum toxin type B, wherein the botulinum toxin type B is to be administered in a dose ratio of 2/3 into the parotid glands and 1/3 into the submandibular glands.

In some embodiments, the use comprises administering botulinum toxin one day to eight weeks prior to an imaging procedure or radiological treatment using one or more radionuclides.

In some embodiments, the use comprises administering the anticholinergic three days, two days, one day, and the same day prior to diagnostic imaging procedure or radiological treatment using one or more radionuclides, and about 7 to 30 days after the diagnostic imaging procedure or after a radiological treatment.

In some embodiments, the use comprises administering the botulinum toxin one day to eight weeks and the anticholinergic three days, two days, one day and the same day prior to a diagnostic imaging procedure or prior to radiological treatment using one or more radionuclides, and about 7 to 30 days after the diagnostic imaging procedure or a radiological treatment.

In some embodiments, the glands are exocrine and/or endocrine glands.

In some embodiments, the gland is a seromucous gland.

In some embodiments, botulinum toxin type A acts by down-regulating the α-1 adrenoreceptors in the glands.

In some embodiments, the use is preferably carried out in a group of patients, which has already undergone anti-androgen therapy or is undergoing anti-androgen therapy during radionuclide therapy.

### Advantages of the Invention

By using botulinum toxin and an anticholinergic in the method of the present invention, salivary and ocular glands are protected. This means that permanent and irreversible side effects such as dry mouth, ulcers in the entire mouth area, dysphagia, risk of aspiration, dental damage are prevented and thus further treatment of these side effects is also prevented. Therefore, the health care system is relieved. In a worst case scenario, severe side effects can result in a dose adjustment or even discontinuation of radioligand therapy. The present invention also prevents these scenarios.

In some embodiments, the method comprises the use of botulinum toxin and an anticholinergic. This further increases the protective effect on the glands. The combination with an anticholinergic leads to an enhancement of the botulinum toxin effect at the parotid gland and submandibular gland and additional to a protective effect at the other salivary glands and ocular glands as long as the respective anticholinergic remains effective. This means that permanent and irreversible side effects such as dry eyes, repeated eye inflammations, visual impairment, loss of visual acuity are prevented in addition to dry mouth, ulcers in the entire mouth area, dysphagia, risk of aspiration and dental damage, and thus also further treatment of these side effects.

### FIGURES

The following Figures show examples of preferred therapy regimens. This means that other therapy regimens are also possible and encompassed by the present invention. This means that monotherapies with botulinum toxin type A or type B also represent exemplary therapy regimens.

FIG. 1: Therapy regimen for the prevention of radiation damage from the use of botulinum toxin type A or type B in the therapeutic use of radioligands (every eight weeks, in this example, four administrations of the radioligand are shown as part of the therapeutic procedure, whereby administrations may occur more than four times)

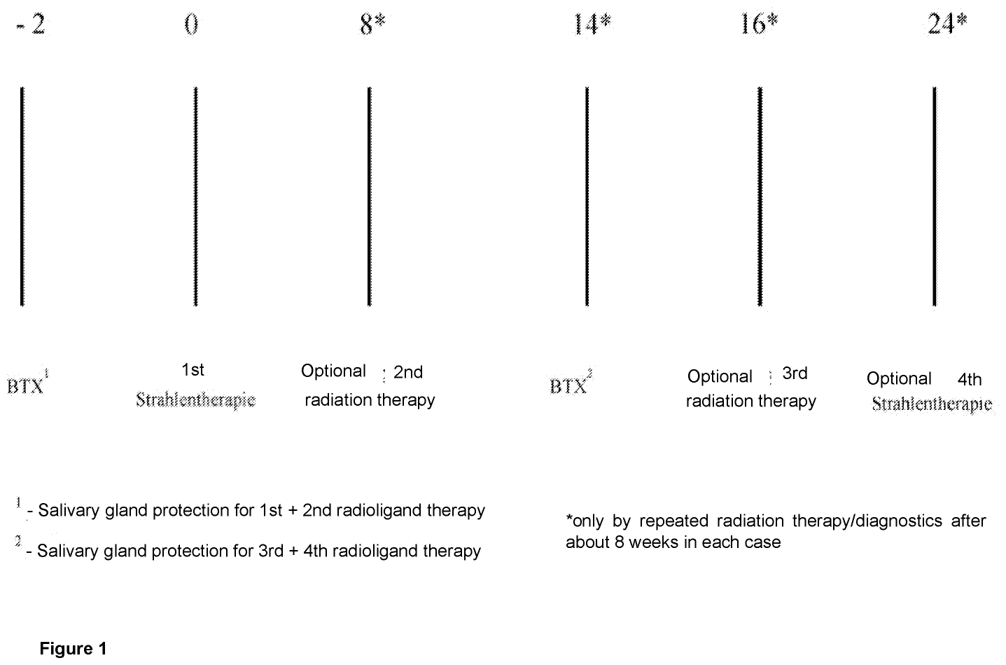

FIG. 2: Therapeutic regimen for the prevention of radiation damage from the use of botulinum toxin type A or B and an anticholinergic in the diagnostic and initial therapeutic use of radioligands.

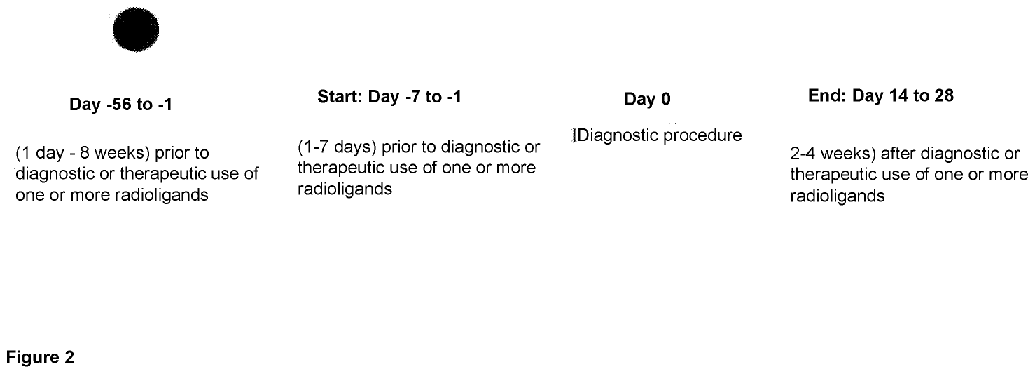

FIG. 3: Therapeutic regimen for the prevention of radiation damage from the use of botulinum toxin type A or type B and an anticholinergic in the therapeutic use of radioligands (every 56 days, in this example three out of four administrations of radioligand are shown as part of the therapeutic procedure).

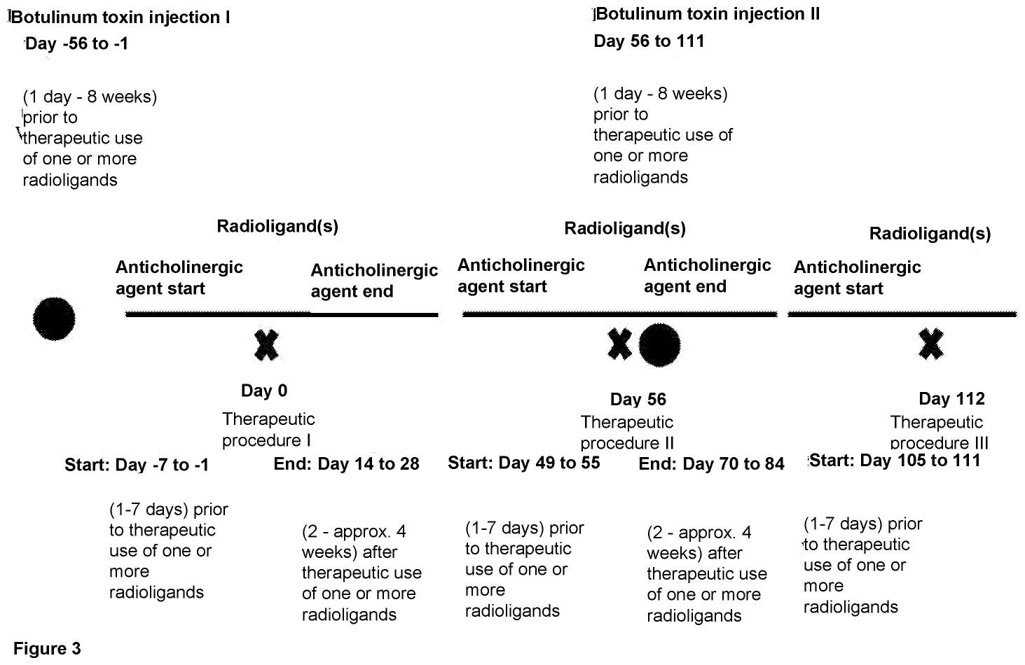

### DETAILED DESCRIPTION

### Definitions

“Botulinum toxin” is a generic term for the family of toxins produced by the bacterium Clostridium botulinum. There are currently seven different known botulinum toxins (A, B, C, D, E, F and G) that differ in terms of their properties. In the present application, botulinum toxin means that all different types are encompassed. There are currently three type A botulinum toxin preparations and one type B botulinum toxin preparation in clinical use.

“Anticholinergic agents” is a collective term for active ingredients that antagonize the effect of the neurotransmitter acetylcholine at the effector cells, whereby the initial release of acetylcholine remains unaffected.

“Prevention” also called prophylaxis, refers to measures to avert adverse events or conditions that could occur with a certain likelihood, if nothing were done. In the context of this application, prevention means avoiding damage or side effects to human glands and other parts of the body which could occur as a result of diagnosis or therapy with radioligands. This damage includes, for example, dry mouth, ulcers in the entire mouth area, dysphagia, risk of aspiration, dental damage, dry eyes, visual impairment and (chronic) inflammation of the eyes and loss of visual acuity.

“Radiation damage” is damage caused to organisms by ionizing radiation. The effect of ionizing radiation on the organism may consist of a number of physical, chemical, biochemical and biological processes. In the context of the present invention, it means damage or side effects caused by the use of radioligands for diagnostics and/or therapy. These damages or side effects include, for example, dry mouth, ulcers throughout mouth, dysphagia, risk of aspiration, dental damage, dry eyes, visual impairment and (chronic) eye inflammation, and loss of visual acuity.

“Radionuclide” is a nuclide if it is unstable and therefore radioactive. Radionuclides of the present invention are radionuclides that are used for medical purposes, especially in diagnostics such as imaging procedures or for therapy.

“Radioligand” is a substance labeled with a radionuclide capable of binding as a ligand to a target protein, such as, for example, a receptor. Such radioligands are used, for example, both in diagnostics and in the therapy of, for example, oncological diseases.

“Glands” are organs, and by extension, individual cells, capable of synthesizing and secreting specific substances. Glands can have serous and mucous parts at the same time, which have an influence on the substance produced. Non-limiting examples of such substances are saliva or tears. Another classification of glands is the division of glands into exocrine and endocrine glands.

“Dose ratio” The amount of a pharmaceutical active ingredient or drug intended to be administered, here the amount of botulinum toxin, is typically referred to as dose. Dose ratios can be expressed as fractions (e.g., 1/3) or as a ratio, such as 1:1, 1:2 etc.

“Cognitive disorders” are memory disorders of any kind, and include mild, moderate and severe cognitive disorders. A mild cognitive disorder or mild cognitive impairment (MCI) is an impairment of the thinking ability that is beyond what is normal for a person's age and education, but does not represent a significant disability in everyday life.

“Reduced life expectancy” Reduced life expectancy is between six and eighteen months, preferably six months from the time of diagnosis.

“High-dose therapy” High-dose therapy is understood to mean the application of a total of between 100 and 300 units of botulinum toxin type A, particularly preferably a total of 150 units of botulinum toxin type A. In this case, between 75 and 150 units, particularly preferably 100 units, are applied to a parotid gland and between 25 and 75 units, more preferably 50 units are applied to a submandibular gland. The high-dose botulinum toxin type A is particularly preferably applied asymmetrically (contralaterally).

“Asymmetric (contralateral) application” In the case of an asymmetric (contralateral) application, the botulinum toxin is injected into a gland and into the gland that is on the opposite side of the body. For example, the botulinum toxin is applied to the right parotid gland and to the (contralateral) left submandibular gland (50 units). This means for example, that the right parotid gland and the left submandibular gland can be selected for injection, or vice versa.

“Dementia” Dementia is a collection of symptoms of various diseases, the main feature of which is a deterioration in several cognitive abilities compared to an earlier state. Dementia can result from various degenerative and non-degenerative diseases of the brain.

“Comorbid” or “multimorbid” means that a patient suffers from another pathology or syndrome in addition to an underlying disease, or from several different diseases at the same time.

### EMBODIMENTS OF THE INVENTION

The method of the present invention is suitable for a variety of uses. It is not only suitable for diagnosing diseases for which radionuclides or radioligands are used, but also for the treatment of diseases for which radionuclides or radioligands are used. Exemplary radionuclides include ³²P, ⁶⁰Co, ⁹⁰Sr, ⁹⁰Y, ¹⁰³Pd, ¹²⁵I, ¹³¹I, ¹³⁷Cs, ¹⁸⁸Re, ¹⁹²Ir, ¹⁹⁸Au, ²²⁶Ra. Diseases include oncological diseases, such as bronchial carcinomas, such as small-cell or large-cell bronchial carcinoma, colon cancer, breast cancer, prostate cancer, liver cancer, pancreatic cancer, bladder cancer, skin cancer, ovarian cancer, cancers of the urogenital tract, adrenal cortex cancer (pheochromocytoma), cancers of the brain, stomach cancer, kidney cancer, uterine cancer, bone cancer, esophageal cancer, oropharyngeal cancers, testicular cancer, thyroid cancer, adrenocortical cancer, gallbladder cancer, small intestinal cancer, anal cancer, pancreatic cancer, bile duct cancer, cervical cancer, uterine body cancer, urethral cancer, laryngeal cancer, bone cancer, Wilms' tumor, plasmacytoma, Hodgkin's lymphoma, non-Hodgkin's lymphoma, or retinoblastoma. Other diseases, which require diagnosis or therapy by means of radioligands are also included.

The present invention relates to botulinum toxin in combination with an anticholinergic for use in a method of preventing radiation damage to the glands, wherein radiation damage is caused by radioligands. Radioligands within the meaning of the invention include all radionuclides that are coupled to a ligand.

In some embodiments, botulinum toxin is used in combination with an anticholinergic for use in a method, wherein the radiation damage is caused by PSMA radioligands. A particularly preferred PSMA radioligand is PSMA-617.

In some embodiments, botulinum toxin is used in combination with an anticholinergic for use in a method, wherein the radiation damage is caused by ²²⁵Ac-PSMA-617, ⁶⁸Ga-PSMA, ¹⁸F-PSMA or ¹⁷⁷Lu-PSMA. In some embodiments, the PSMA is coupled to other diagnostically or therapeutically suitable radionuclides, such as ¹¹¹ In.

In some embodiments, the botulinum toxin is selected from the group consisting of botulinum toxin types A, B, C, D, E, F and G. In some embodiments, a botulinum toxin type A is preferred. In some embodiments, a botulinum toxin type B is preferred. In further embodiments, a combination of the different botulinum toxin types is preferred, such as type A and type B, type A and E, type A and F, type Band E, type B and F. Modified, recombinant and synthetic botulinum toxins are also included. Modified botulinum toxins include amino acid substitutions, deletions or insertions made in codons of a polynucleotide, which in turn encodes the modified polypeptide. Also included are chemical modifications such as PEGylation of the botulinum toxin or phosphorylation of the amino acids. Recombinant botulinum toxins are recombinantly produced botulinum toxins; methods of recombinant DNA technology for the production of such recombinant botulinum toxins are well known. Synthetic botulinum toxins are polypeptides with a defined sequence. Synthetic botulinum toxins can represent the exact copy of the naturally occurring botulinum toxin or can be produced in various modifications. For example, synthetic botulinum toxins can comprise unnatural amino acids or modification of the peptide backbone.

In some embodiments, the botulinum toxin comprises between 1 and 10,000 units, preferably between 10 and 5,000 units, and most preferably between 20 and 4,000 units.

In some embodiments, the botulinum toxin comprises between 1 unit and 1,500, preferably between 10 and 1,000 units, and most preferably between 20 and 900 units of type A.

In some embodiments, the botulinum toxin comprises between 100 and 10,000 units, preferably between 250 and 5,000 units, and most preferably between 500 and 4,000 units of type B.

In some embodiments, the botulinum toxin is a botulinum toxin type E or type F.

In some embodiments, the botulinum toxin comprises between 1 unit and 10,000 units of type E or type F.

In some embodiments, the anticholinergic is selected from the group consisting of tropicamide, atropine, scopolamine, glycopyrrolate, amitriptyline, clonidine, ipratropium bromide, and trihexyphenidyl. Scopolamine is particularly preferred here. In some embodiments, the anticholinergic is administered transdermally, orally, or intravenously. Preferably, the anticholinergic is administered transdermally. Most preferably, scopolamine is administered transdermally.

In some embodiments, the botulinum toxin is botulinum toxin type B for the prevention of radiation damage to the parotid, submandibular, sublingual, minor salivary and ocular glands, wherein the radiation damage is caused by radioligands. In some embodiments, botulinum toxin type B is used in a method in which patients undergoing radionuclide diagnostics or treatment suffer from a cognitive disorder. In some embodiments, botulinum toxin type B is used in a method in which patients undergoing radionuclide diagnostics or treatment suffer from cardiac arrhythmia. In a preferred embodiment, botulinum toxin type B is used in a method in which patients undergoing radionuclide diagnostics or treatment suffer from cognitive impairment and cardiac arrhythmia.

In some embodiments, the botulinum toxin is botulinum toxin type A for lowering aquaporin-mediated radionuclide uptake in submandibular and salivary gland cells. In some embodiments, botulinum toxin type A is used in a method in which patients undergoing radionuclide diagnostics or treatment suffer from cognitive impairment. In some embodiments, botulinum toxin type A is used in a method in which patients undergoing radionuclide diagnostics or treatment suffer from cardiac arrhythmia. In another embodiment, botulinum toxin type A is used in a method in which patients undergoing radionuclide diagnostics or treatment suffer from cognitive impairment and cardiac arrhythmia.

In one embodiment, the botulinum toxin is botulinum toxin type A, wherein the botulinum toxin type A is to be administered in a dose ratio of 1:1, 2:1, 3:1, 4:1, 5:1 to the parotid and submandibular glands. In a particularly preferred embodiment, the botulinum toxin is botulinum toxin type A, wherein the botulinum toxin type A is to be administered in a dose ratio of 2/3 into the parotid glands and 1/3 into the submandibular glands, in other words, in a ratio of 2:1. In a particularly preferred embodiment, the botulinum toxin type A is administered in high doses, or in other words, as “high dose therapy.” A total of between 100 and 300 units of botulinum toxin type A are applied, particularly preferably 150 units of botulinum toxin type A. Between 75 and 150 units, particularly preferably 100 units, are applied in a parotid gland and between 25 and 75 units, particularly preferably 50 units are applied in a submandibular gland. The high-dose botulinum toxin type A is particularly preferably applied contralaterally (asymmetrically). The botulinum toxin type A was applied asymmetrically (100 units) in the right parotid gland and in the contralateral, left submandibular gland (50 units). This means that, for example, the right parotid gland and the left submandibular gland can be selected for injection, or vice versa.

In an embodiment, the botulinum toxin is botulinum toxin type B, wherein the botulinum toxin type B is to be administered in a dose ratio of 1:1, 2:1, 3:1, 4:1, 5:1 to the parotid and submandibular glands. In a particularly preferred embodiment, the botulinum toxin is botulinum toxin type B, wherein the botulinum toxin type B is to be administered in a dose ratio of 2/3 into the parotid glands and 1/3 into the submandibular glands, i.e., in a ratio of 2:1.

In some embodiments, the use includes administering botulinum toxin one day to sixteen weeks prior to an imaging procedure or radiological treatment using one or more radionuclides. In preferred embodiments, the use comprises administering botulinum toxin one day to eight weeks prior to an imaging procedure or radiological treatment using one or more radionuclides. In this case, the time of administration depends on the botulinum toxin type used: Type E and type F are administered one day to eight weeks prior to an imaging procedure or radiological treatment, more preferably one day to four weeks, even more preferably one day to two weeks, most preferably 1 day to 7 days prior to an imaging procedure or prior to radiological treatment using one or more radionuclides. Types A and B are administered three days to twelve weeks prior to an imaging procedure or radiological treatment, preferably three days to four weeks prior to an imaging procedure or radiological treatment using one or more radionuclides, also preferably between two and four Weeks, most preferably two weeks, prior to an imaging procedure or radiological treatment.

In some embodiments, the use comprises administering the anticholinergic seven days, six days, five days, four days, three days, two days, one day, and on the same day prior to a diagnostic imaging procedure or prior to radiological treatment using one or more radionuclides, and up to about 14 to 28 days after the diagnostic imaging procedure or after a radiological treatment. In some embodiments, the use comprises administering the anticholinergic seven days, six days, five days, four days, three days, two days, one day and on the same day prior to a diagnostic imaging procedure or prior to radiological treatment using one or more radionuclides, and up to about 14 to 28 days after the diagnostic imaging procedure or after a radiological treatment. As previously mentioned, the anticholinergic may have various dosage forms. If the anticholinergic in the present invention is applied transdermally, it can be administered daily, every other day, every third day or even every fourth day.

In some embodiments, the use comprises administering the botulinum toxin four weeks and the anticholinergic seven days, six days, five days, four days, three days, two days, one day and on the same day prior to a diagnostic imaging procedure or prior to radiological treatment using one or more radionuclides, and up to 28 days after the diagnostic imaging procedure or a after radiological treatment. In preferred embodiments, the use includes administering the botulinum toxin two weeks and the anticholinergic seven days, six days, five days, four days, three days, two days, one day and on the same day prior to a diagnostic imaging procedure or prior to radiological treatment using one or more radionuclides, and up to 28 days after the diagnostic imaging procedure or after a radiological treatment. As previously mentioned, the anticholinergic may have various dosage forms. If the anticholinergic in the present invention is applied transdermally, it can be administered daily, every other day, every third day or even every fourth day. In a preferred embodiment, patients treated with both botulinum toxin and an anticholinergic have a mean age of between 50 and 75 years.

In some embodiments, the glands are exocrine and/or endocrine glands.

In some embodiments, the glands are selected from the group consisting of parotid, submandibular, sublingual, minor salivary, and ocular glands. As the listing suggests, the glands can be selected individually or in any combination. Moreover, a person skilled in the art knows that some glands are present on one or both sides, and can be combined accordingly. Therefore, the various glands can also be selected, for example, asymmetrically (contralaterally), that is to say, the right parotid gland and the left submandibular gland can, for example, be selected, or vice versa. In one embodiment, the botulinum toxin can be botulinum toxin type A or type B.

In a particularly preferred embodiment, the botulinum toxin type A is administered in high doses, or in other words, as “high dose therapy.” In this case, a total of between 100 and 300 units of botulinum toxin type A is applied, particularly preferably 150 units of botulinum toxin type A. In this embodiment, the botulinum toxin type A can be administered alone (“monotherapy”) or in combination with other agents, such as, for example, an anticholinergic. In a particularly preferred embodiment, botulinum toxin type A is applied alone. In one embodiment, between one and four glands are injected, and in a preferred embodiment, two glands are injected. Between 75 and 150 units, particularly preferably 100 units, are applied to a parotid gland, and between 25 and 75 units, particularly preferably, 50 units are applied to a submandibular gland. The high-dose botulinum toxin type A is particularly preferably applied asymmetrically (contralaterally). The botulinum toxin type A was applied asymmetrically (100units) to the right parotid gland and to the contralateral, left submandibular gland (50 units). The advantage of this application is that two of the four large salivary glands are protected at a maximum, without any side effects, and the patient initially still maintains sufficient saliva production through the two other glands during the initial radioligand administration, until the two untreated glands are irreversibly damaged, which often only fully occurs with the second or subsequent radioligand administration. However, high-dose therapy of all four glands is excluded, due to the risk of side effects in the form of dysphagia.

This type of selecting and combining the salivary glands on one or both sides is particularly advantageous for co-morbid and multi-morbid patients, patients aged>75 years, patients suffering from mild cognitive impairment (MCI) or dementia, or patients with reduced life expectancy. These patient groups can be freely combinable and permutable, for example a patient can be >75 years old, suffer from mild cognitive impairment and, due to a disease, require diagnostics or therapy with a radioligand. It should also be emphasized that in these patient groups, an anticholinergic should ideally be avoided in order to avoid anticholinergic side effects. Reduced life expectancy pursuant to the invention is from six to eighteen months, preferably six months. Therapy with fewer injections, albeit with a higher dose, is particularly advantageous for this group of patients. Any side effects in all patients can also be avoided by selecting and combining the salivary glands on one or both sides.

In some embodiments, the gland is a submandibular gland.

In some embodiments, the gland is a seromucous gland. In other embodiments, the gland is a mucous gland, or a serous gland.

In some embodiments, botulinum toxin type A acts by down-regulating the α-1 adrenoreceptors in the glands. In some embodiments, the botulinum toxin also acts by down-regulating the α-1 adrenoreceptors in the glands, while other mechanisms are also effective.

In some embodiments, the use is preferably carried out in a group of patients who have already undergone anti-androgen therapy. In some embodiments, the use is performed in a group of patients that has not undergone anti-androgen therapy. In further embodiments, the application is carried out in a group of patients undergoing anti-androgen therapy during radioligand diagnostics or therapy.

In some embodiments, botulinum toxin type A acts to downregulate aquaporin-mediated radionuclide uptake in the parotid and submandibular glands.

### EXAMPLES

The present invention is described in detail by the following non-limiting examples:

### Example 1: Prevention of Radiation Damage from the Use of Botulinum Toxin Type A and an Anticholinergic in the Diagnostic Use of Radioligands Using the Example of ⁶⁸Ga-PSMA

Two weeks prior to the diagnostic use of ⁶⁸Ga-PSMA in an imaging procedure, a total of between 20 and 900 units of botulinum toxin type A, 150 units when using Xeomin® or BOTOX® as an example, depending on the specific botulinum toxin type A preparation, are applied to the relevant glands, such as the mandibular gland on one or both sides and/or to the parotid glands on one or both sides. Three days prior to the imaging procedure using ⁶⁸Ga-PSMA, additional administration of the anticholinergic is initiated. The anticholinergic, in this case scopolamine, is applied every three days in the form of one or two transdermal patches. In this case, about 1 mg of scopolamine is released into the systemic circulation at an almost constant rate within 72 hours. Alternatively, the anticholinergic is administered orally every day (two days prior to the diagnostic procedure, one day prior to the diagnostic procedure, on the day of the diagnostic procedure) and up to 21 days after the diagnostic procedure. This approach also protects the sublingual gland, the minor salivary glands and the ocular glands. After two weeks (day 0), the imaging procedure with ⁶⁸Ga-PSMA takes place, for example, to obtain follow-up imaging of a prostate carcinoma patient. The uptake of ⁶⁸Ga-PSMA in the glands treated with Botulinum toxin is significantly reduced as opposed to, for example, brain parenchyma. This reduced uptake protects the respective glands and allows them to function normally, thus avoiding persistent side effects such as dry mouth, dry eyes and similar symptoms.

### Example 2: Prevention of Radiation Damage from the Use of Botulinum Toxin Type B and an Anticholinergic in the Diagnostic Use of Radioligands Using the Example of ⁶⁸Ga-PSMA

Two weeks prior to the diagnostic use of ⁶⁸Ga-PSMA in an imaging procedure, a total of 3,000 units of botulinum toxin type B, for example MYOBLOC®, are applied to the desired glands, such as the submandibular gland on one or both sides and/or in the parotid glands on one or both sides. Three days prior to the imaging procedure using ⁶⁸Ga-PSMA, the additional administration of the anticholinergic is initiated. The anticholinergic, here scopolamine, is applied in the form of one or two transdermal patches every three days. In this case, about 1 mg of scopolamine is released into the systemic circulation at an almost constant rate within 72 hours. Alternatively, the anticholinergic is administered orally every day (two days prior to the diagnostic procedure, one day prior to the diagnostic procedure, on the day of the diagnostic procedure) and 14 days after the diagnostic procedure. This approach additionally protects the sublingual gland, the minor salivary glands and the ocular glands. After two weeks (day 0), the imaging procedure with ⁶⁸Ga-PSMA takes place, for example, to obtain follow-up imaging of a prostate carcinoma patient. The uptake of ⁶⁸Ga-PSMA in the glands treated with botulinum toxin is significantly reduced in contrast to, for example, brain parenchyma. This reduced uptake protects the respective glands and allows them to function normally, thus avoiding persistent side effects such as dry mouth, dry eyes and similar symptoms.

### Example 3: Prevention of Radiation Damage from the Use of Botulinum Toxin and an Anticholinergic in the Diagnostic Use of Radioligands Using the Example of ¹⁸F-PSMA and Botulinum Toxin Type A

Two weeks prior to the diagnostic use of ¹⁸F-PSMA in an imaging procedure, a total of between 20 and 900 units of botulinum toxin type A, 150 units when using Xeomin® or BOTOX® as an example, depending on the specific botulinum toxin type A preparation, are applied to the desired glands, such as the submandibular gland on one or both sides and/or the parotid glands on one or both sides. Three days prior to the imaging procedure using ¹⁸F-PSMA, additionally the anticholinergic is initiated. The anticholinergic, here scopolamine, is applied in the form of one or two transdermal patches every three days. In this case about 1 mg of scopolamine is released into the systemic circulation at an almost constant rate within 72 hours. Alternatively, the anticholinergic is administered orally every day (two days prior to the diagnostic procedure, one day prior to the diagnostic procedure, on the day of the diagnostic procedure) and up to 21 days after the diagnostic procedure. This approach also protects the sublingual gland, the minor salivary glands and the ocular glands. After two weeks (day 0), the imaging procedure with ¹⁸F-PSMA takes place, for example, to obtain follow-up imaging of a prostate carcinoma patient. The uptake of ¹⁸F-PSMA in the treated glands is significantly reduced in contrast to, for example, the brain parenchyma. This reduced uptake protects the respective glands and allows them to function normally, thus avoiding side effects such as dry mouth, dry eyes and similar symptoms.

### Example 4: Prevention of Radiation Damage From the Use of Botulinum Toxin and an Anticholinergic in the Diagnostic Use of Radioligands Using the Example of ¹⁸F-PSMA and Botulinum Toxin Type B

Two weeks prior to the diagnostic use of ⁶⁸Ga-PSMA in an imaging procedure, a total of 3,000 units of botulinum toxin type B, for example MYOBLOCO, are applied to the desired glands, such as the submandibular gland on one or both sides and/or in the parotid glands on one or both sides. Three days prior to the imaging procedure using ¹⁸F-PSMA, additionally the anticholinergic is initiated. The anticholinergic, here scopolamine, is applied in the form of one or two transdermal patches every three days. In this case, about 1 mg of scopolamine is released into the systemic circulation at an almost constant rate within 72 hours. Alternatively, the anticholinergic is administered orally every day (two days prior to the diagnostic procedure, one day prior to the diagnostic procedure, on the day of the diagnostic procedure) and up to 21 days after the diagnostic procedure. This approach additionally protects the sublingual gland, the minor salivary glands and the ocular glands. After two weeks (day 0) the imaging procedure with ¹⁸F-PSMA takes place, for example to obtain follow-up imaging in a prostate carcinoma patient. The uptake of ¹⁸F-PSMA in the treated glands is significantly reduced in contrast to, for example, the brain parenchyma. This reduced uptake protects the respective glands and allows them to function normally, thus avoiding side effects such as dry mouth, dry eyes and similar symptoms.

### Example 5: Prevention of Radiation Damage from the Use of Botulinum Toxin Type A and an Anticholinergic in the Therapeutic Use of Radioligands Using the Example of ²²⁵ Ac-PSMA-617

Two weeks prior to the first therapeutic use of ²²⁵Ac-PSMA-617, a total of between 20 and 900 units of botulinum toxin type A, 150 units when using Xeomin® or BOTOX® as an example, depending on the specific botulinum toxin type A preparation, are applied to the desired glands, such as the submandibular glands on one or both sides and/or to the parotid glands on one or both sides. Three days prior to therapy using ²²⁵Ac-PSMA-617, additionally the anticholinergic is initiated. The anticholinergic, here scopolamine, is applied in the form of one or two transdermal patches every three days. In this case, about 1 mg of scopolamine is released into the systemic circulation at an almost constant rate. Alternatively, the anticholinergic is administered orally every day (two days prior to the therapeutic procedure, one day prior to the therapeutic procedure, on the day of the therapeutic procedure) and up to 21 days after the therapeutic procedure. This approach also protects the sublingual gland, the minor salivary glands and the ocular glands. After two weeks (day 0), the first administration of ²²⁵Ac-PSMA-617 (100 kBq/kg) takes place to initiate the treatment of a prostate carcinoma patient. The uptake of ²²⁵Ac-PSMA-617 in the treated glands is significantly reduced compared to, for example, the brain parenchyma. This reduced uptake protects the respective glands and allows them to function normally, thus avoiding severe, persistent and irreversible side effects such as, for example, dehydration of the eyes, which severely restricts the quality of life, and chronic severe inflammation of the eyes and lid margin, and dysphagia, associated weight loss and inflammation of oral mucosa and dental damage and similar symptoms are prevented. After about eight weeks, another administration of ²²⁵Ac-PSMA-617 (100 kBq/kg) takes place. Two weeks prior to another therapeutic procedure, during the further course of therapy with ²²⁵Ac-PSMA-617, a total of between 20 and 900 units of botulinum toxin type A, between 50 and 150 units when using Xeomin® or BOTOX® as an example, are administered depending on the specific botulinum toxin type A preparation and depending on the extent of the effect of the previous botulinum toxin injection. The anticholinergic is additionally initiated three days prior to therapy using ²²⁵Ac-PSMA-617. This agent is administered every day (two days prior to the therapeutic procedure, one day prior to the therapeutic procedure, on the day of the therapeutic procedure) and up to 21 days after the therapeutic procedure. At the same time, the therapy cycles with ²²⁵Ac-PSMA-617 take place, which take place every eight weeks. Approximately four cycles of therapy with ²²⁵ Ac-PSMA-617 are expected to be required.

### Example 6: Prevention of Radiation Damage from the Ise of Botulinum Toxin Type B and an Anticholinergic in the Therapeutic Use of Radioligands Using the Example of ²²⁵Ac-PSMA-617

Two weeks prior to the first therapeutic use of ²²⁵Ac-PSMA-617, a total of 3,000 units of botulinum toxin type B, for example MYOBLOC®, are applied to the desired glands, such as the submandibular gland on one or both sides and/or the parotid glands on one or both sides. Three days prior to therapy using ²²⁵Ac-PSMA-617, the anticholinergic is additionally initiated. The anticholinergic, here scopolamine, is applied in the form of one or two transdermal patches every three days. About 1 mg of scopolamine is released into the systemic circulation at an almost constant rate. Alternatively, the anticholinergic is administered orally every day (two days prior to the therapeutic procedure, one day prior to the therapeutic procedure, on the day of the therapeutic procedure) and up to 21 days after the therapeutic procedure. This approach additionally protects the sublingual gland, the minor salivary glands and the ocular glands. After two weeks (day 0), the first administration of ²²⁵Ac-PSMA-617 (100 kBq/kg) takes place to start the treatment of a prostate carcinoma patient. The uptake of ²²⁵Ac-PSMA-617 in the treated glands is significantly reduced compared to, for example, the brain parenchyma. This reduced uptake protects the respective glands and allows them to function normally, thus avoiding severe, persistent and irreversible side effects such as, for example, dehydration of the eyes, which severely restricts the quality of life, and chronic severe inflammation of the eyes and lid margin, and dysphagia, associated weight loss and inflammation of oral mucosa and dental damage and similar symptoms are prevented. After about eight weeks, another administration of ²²⁵Ac-PSMA-617 (100 kBq/kg) takes place. Two weeks prior to the therapeutic procedure, during the further course of therapy with ²²⁵Ac-PSMA-617, a total of between 1,000 and 3,000 units of botulinum toxin type B are administered into the desired glands depending on the extent of the effect of the previous botulinum toxin injection. The anticholinergic is additionally initiated three days prior to therapy using ²²⁵Ac-PSMA-617. This agent is administered every day (two days prior to the therapeutic procedure, one day prior to the therapeutic procedure, on the day of the therapeutic procedure) and up to 21 days after the therapeutic procedure. At the same time, the therapy cycles with ²²⁵Ac-PSMA-617 take place, which take place every eight weeks. Approximately four cycles of therapy with ²²⁵Ac-PSMA-617 are expected to be required.

### Example 7: Prevention of Radiation Damage from the Use of Botulinum Toxin Type A and an Anticholinergic in the Therapeutic Use of Radioligands Using the Example of ¹⁷⁷Lu-PSMA

A total of between 20 and 900 units of botulinum toxin type A, 150 units when using Xeomin® or BOTOX® as an example, depending on the specific botulinum toxin type A preparation, is applied to the desired glands, such as the submandibular gland on one or both sides and/or the parotid glands on one or both kobo sides. Three days prior to therapy using ¹⁷⁷Lu-PSMA, additionally, the anticholinergic is additionally administered. The anticholinergic, here scopolamine, is applied in the form of one or two transdermal patches every three days. In this case, about 1 mg of scopolamine is released into the systemic circulation at an almost constant rate within 72 hours. Alternatively, the anticholinergic is administered orally every day (two days prior to the therapeutic procedure, one day prior to the therapeutic procedure, on the day of the therapeutic procedure) and up to 21 days after the therapeutic procedure. This approach additionally protects the sublingual gland, the minor salivary glands and the ocular glands. After two weeks (day 0), the first administration of ¹⁷⁷LU-PSMA (6 GBq/kg) takes place to start the treatment of a prostate carcinoma patient. The uptake of ¹⁷⁷Lu-PSMA in the treated glands is significantly reduced in contrast to, for example, the brain parenchyma. This reduced uptake protects the respective glands and allows them to function normally, thus avoiding severe, persistent and irreversible side effects such as dry eyes, which severely restrict the quality of life, and chronic severe inflammation of the eyes and lid margin, dysphagia, associated weight loss, inflammation of oral mucosa and dental damage and similar symptoms are prevented. After about eight weeks, another administration of ¹⁷⁷Lu-PSMA (100 kBq/kg) takes place. One day to eight weeks prior to the therapeutic procedure, during the further course of therapy with ¹⁷⁷Lu-PSMA, a total of between 20 and 900 units of botulinum toxin type A, between 50 and 150 units when using Xeomin® or BOTOX® as an example, are applied depending on the specific botulinum toxin type A preparation and depending on the extent of the effect of the previous botulinum toxin injection, in the desired glands, such as the submandibular gland on one or both sides and/or to the parotid glands on one or both sides. The anticholinergic is additionally administered three days prior to therapy using ¹⁷⁷Lu-PSMA. This agent is administered every day (two days prior to the therapeutic procedure, one day prior to the therapeutic procedure, on the day of the therapeutic procedure) and up to 21 days after the therapeutic procedure. At the same time, the therapy cycles with ¹⁷⁷Lu-PSMA take place every eight weeks. Approximately four therapy cycles with ¹⁷⁷Lu-PSMA are expected to be required.

### Example 8: Prevention of Radiation Damage from the Use of Botulinum Toxin Type

B and an anticholinergic in the therapeutic use of radioligands using the example of ¹⁷⁷Lu-PSMA

Two weeks prior to the first therapeutic use of ¹⁷⁷Lu-PSMA, a total of 3,000 units of botulinum toxin type B, for example MYOBLOC®, are applied to the desired glands, such as the submandibular gland on one or both sides and/or the parotid glands on one or both sides. Three days prior to therapy using ¹⁷⁷LU-PSMA, the anticholinergic is additionally administered. The anticholinergic, here scopolamine, is applied in the form of one or two transdermal patches every three days. About 1 mg of scopolamine is released into the systemic circulation at an almost constant rate within 72 hours. Alternatively, the anticholinergic is administered orally every day (two days prior to the therapeutic procedure, one day prior to the therapeutic procedure, on the day of the therapeutic procedure) and up to 21 days after the therapeutic procedure. This approach additionally protects the sublingual gland, the minor salivary glands and the ocular glands. After two weeks (day 0), the first administration of ¹⁷⁷Lu-PSMA (6 GBq/kg) takes place to start the treatment of a prostate carcinoma patient. The uptake of ¹⁷⁷Lu-PSMA in the treated glands is significantly reduced in contrast to, for example, the brain parenchyma. This reduced uptake protects the respective glands and allows them to function normally, thus avoiding severe, persistent and irreversible side effects such as, for example, dehydration of the eyes, which severely restricts the quality of life, and chronic severe inflammation of the eyes and lid margin and, dysphagia, associated weight loss and inflammation of oral mucosa and dental damage and similar symptoms are prevented. After about eight weeks, another dose of ¹⁷⁷Lu-PMSA (100 kBq/kg) is administered. Two weeks prior to the therapeutic procedure, during the further course of therapy with ¹⁷⁷Lu-PSMA, a total of between 1,000 and 3,000 units of botulinum toxin type B, depending on the extent of the effect of the previous botulinum toxin injection, are applied to the desired glands, such as the submandibular gland on one or both sides and/or the parotid glands on one or both sides. The anticholinergic is additionally administered three days prior to therapy using ¹⁷⁷Lu-PSMA. This agent is administered every day (two days prior to the therapeutic procedure, one day prior to the therapeutic procedure, on the day of the therapeutic procedure) and up to 21 days after the therapeutic procedure. At the same time, the therapy cycles with ¹⁷⁷Lu-PSMA take place every eight weeks. Approximately four therapy cycles with ¹⁷⁷Lu-PSMA are expected to be required.

### Example 9: Prevention of Radiation Damage from the Use of Botulinum Toxin Type A in the Diagnostic Use of Radioligands Using the Example of ⁶⁸Ga-PSMA

Two weeks prior to the diagnostic use of ⁶⁸Ga-PSMA in an imaging procedure, a total of between 20 and 900 units of botulinum toxin type A, a total of 150 units when using Xeomin® or BOTOX® as an example, depending on the specific botulinum toxin type A preparation, are applied. The botulinum toxin type A is applied in a fixed dose ratio of 2/3 (parotid gland) to 1/3 (submandibular gland), i.e., 50 units of botulinum toxin type A are applied to each of the two parotid glands and 25 units of botulinum toxin type A are applied to each of the two submandibular glands. After two weeks (day 0), the imaging procedure takes place with ⁶⁸Ga-PSMA, for example, to obtain follow-up imaging of a prostate carcinoma patient. The uptake of ⁶⁸Ga-PSMA in the glands treated with botulinum toxin is significantly reduced in contrast to, for example, brain parenchyma. This reduced uptake protects the respective glands and allows them to function normally, thus avoiding persistent side effects such as dry mouth, dry eyes and similar symptoms.

### Example 10: Prevention of Radiation Damage from the Use of Botulinum Toxin Type B in the Diagnostic Use of Radioligands Using the Example of ⁶⁸Ga-PSMA

Two weeks prior to the diagnostic use of ⁶⁸Ga-PSMA in an imaging procedure, a total of 3,000 units of botulinum toxin type B, for example MYOBLOC®, are applied. The botulinum toxin type B is applied in a fixed dose ratio of 2/3 (parotid gland) to 1/3 (submandibular gland). Assuming a total of 3,000 units, this means that 1,000 units of botulinum toxin type B are applied to each of the two parotid glands and 500 units of botulinum toxin type B are applied to each of the two submandibular glands. After two weeks (day 0), the imaging procedure takes place with ⁶⁸Ga-PSMA, for example, to obtain follow-up imaging of a prostate carcinoma patient. The uptake of ⁶⁸Ga-PSMA in the glands treated with botulinum toxin is significantly reduced in contrast to, for example, brain parenchyma. This reduced uptake protects the respective glands and allows them to function normally, thus avoiding persistent side effects such as dry mouth, dry eyes and similar symptoms.

### Example 11: Prevention of Radiation Damage from the Use of Botulinum Toxin Type A in the Therapeutic Use of Radioligands Using the Example of ²²⁵Ac-PSMA-617

Two weeks prior to the therapeutic use of ²²⁵Ac-PSMA-617 in an imaging procedure, a total of between 20 and 900 units of botulinum toxin type A, 150 units when using the example of Xeomin® or BOTOX®, depending on the specific botulinum toxin type A preparation, are applied. The botulinum toxin type A (Xeomin® or BOTOX®) is applied in a fixed dose ratio of 2/3 (parotid gland) to 1/3 (submandibular gland), i.e., 50 units of botulinum toxin type A are applied to each of the two parotid glands and 25 units of botulinum toxin type A are applied to each of the two submandibular glands. After two weeks (day 0), the first administration of ²²⁵Ac-PSMA-617 (100 kBq/kg) takes place to start the treatment of a prostate carcinoma patient. The uptake of ²²⁵Ac-PSMA-617 in the treated glands is significantly reduced compared to, for example, the brain parenchyma. This reduced uptake protects the respective glands and allows them to function normally, thus avoiding severe, persistent and irreversible side effects such as dryness of the eyes, which severely restricts the quality of life, and chronic severe inflammation of the eyes and lid margin, and dysphagia, associated weight loss and inflammation of oral mucosa and dental damage and similar symptoms are prevented. After about eight weeks, another administration of ²²⁵Ac-PSMA-617 (100 kBq/kg) takes place. In the further course of therapy with ²²⁵Ac-PSMA-617, a total of 20 to 900 units of botulinum toxin type A are administered two weeks prior to the therapeutic procedure, a total of 50 to 150 units when using Xeomin® or BOTOX® as an example, depending on the extent of the effect of the previous botulinum toxin injection. The botulinum toxin type A (Xeomin® or BOTOX®) is administered in a fixed dose ratio of 2/3 (parotid gland) to 1/3 (submandibular gland). At the same time, the therapy cycles with ²²⁵Ac-PSMA-617 take place every eight weeks. Approximately four cycles of therapy with ²²⁵Ac-PSMA-617 are expected to be required.

### Example 12: Prevention of Radiation Damage from the Use of Botulinum Toxin Type B in the Therapeutic Use of Radioligands Using the Example of ²²⁵Ac-PSMA-617

Two weeks prior to the therapeutic use of ²²⁵Ac-PSMA-617 in an imaging procedure, a total of between 3,000 units of botulinum toxin type B, for example MYOBLOC®, are applied. Botulinum toxin type B is applied in a fixed dose ratio of 2/3 (parotid gland) to 1/3 (submandibular gland). Assuming a total of 3,000 units, this means that 1,000 units of botulinum toxin type B are administered in each of the two parotid glands and 500 units of botulinum toxin type B are administered in each of the two submandibular glands. After two weeks (day 0), the first administration of ²²⁵Ac-PSMA-617 (100 kBq/kg) takes place to begin the treatment of a prostate carcinoma patient. The uptake of ²²⁵Ac-PSMA-617 in the treated glands is significantly reduced compared to, for example, the brain parenchyma. This reduced uptake protects the respective glands and allows them to function normally, thus avoiding severe, persistent and irreversible side effects, such as dryness of the eyes, which severely restricts the quality of life, and chronic severe inflammation of the eyes and lid margin, and dysphagia, associated weight loss and inflammation of oral mucosa and dental damage and similar symptoms are prevented. After about eight weeks, another administration of ²²⁵Ac-PSMA-617 (100 kBq/kg) is done. In the further course of therapy with ²²⁵Ac-PSMA-617, a total of between 1,000 and 3,000 units of botulinum toxin type B, depending on the extent of the effect of the previous botulinum toxin injection, are administered two weeks prior to the therapeutic procedure. In parallel, therapy cycles with ²²⁵Ac-PSMA-617 take place every eight weeks. It is anticipated that approximately four cycles of therapy with ²²⁵Ac-PSMA-617 are to be required.

### Example 13: Prevention of Radiation Damage Through the Use of Botulinum Toxin Type A and an Anticholinergic in the Therapeutic Use of ¹⁷⁷Lu-PSMA-617

Two weeks (14 days) prior to the therapeutic use of ¹⁷⁷Lu-PSMA-617, a total of 120 units of botulinum toxin type A (Xeomin®) were applied. The botulinum toxin type A was applied (40 units each) to the parotid glands on both sides and the submandibular glands (20 units each).

The anticholinergic was additionally administered three days prior to therapy using ¹⁷⁷Lu-PSMA-617. The anticholinergic, here scopolamine, is applied in the form of a transdermal patch every three days. In this process, about 1 mg of scopolamine is released into the systemic circulation at an almost constant rate within 72 hours. Said scopolamine patch was replaced on day 0 and day 3 of ¹⁷⁷Lu-PSMA-617 therapy, such that the patient was treated with additional scopolamine for up to six days after therapy. This additionally protects the sublingual gland, the minor salivary glands, and the ocular glands.

Immediately prior to the first ¹⁷⁷Lu-PSMA-617 therapy/day 0, saliva production was normal. In this case, the saliva was 5.7 grams in the Saxon test, with the normal value being ≥2.75 grams of saliva production after two minutes of chewing. After two weeks (day 0), the first administration of ¹⁷⁷Lu-PSMA-617 (6 GBq/kg) took place to begin the treatment of a prostate carcinoma patient.

After about eight weeks (day 56), another administration of ¹⁷⁷Lu-PSMA-617 (6 GBq/kg) took place. After completion of the treatment (two therapy cycles on days 0 and 56) and a follow-up period of approx. eight weeks, on day 108 normal salivary production remained unchanged. Here, the saliva in the Saxon test was 5.5 grams, with the normal value being≥2.75 grams of saliva production after two minutes of chewing. Difficulty swallowing did not occur. Dryness of the eyes was not reported.

### Example 14: Prevention of Radiation Damage from the Use of Botulinum Toxin Type A in the Therapeutic Use of ¹⁷⁷Lu-PSMA-617

Two and a half weeks (18 days) prior to the therapeutic use of ¹⁷⁷Lu-PSMA-617, a total of 150 units of botulinum toxin type A (Xeomin®) were applied. The botulinum toxin type A was applied asymmetrically (100 units) into the right parotid gland and into the contralateral, left submandibular gland (50 units).

Immediately prior to the first ¹⁷⁷Lu-PSMA-617 therapy/day 0, saliva production was normal. In this case, the saliva in the Saxon test was 4.6 grams, with the normal value being ≥2.75 grams of saliva production after two minutes of chewing. After two and a half weeks (day 0), the first administration of ¹⁷⁷Lu-PSMA-617 (6 GBq/kg) took place to start the treatment of a prostate carcinoma patient.

After about eight weeks (day 56) another administration of ¹⁷⁷Lu-PSMA-617 (6GBq/kg) took place. After completion of the treatment (two therapy cycles on days 0 and 56) and a follow-up period of approx. eight weeks, on day 116 normal salivary production remained unchanged. Here, the saliva in the Saxon test was 4.3 grams, with the standard value being≥2.75 grams of saliva production after two minutes of chewing. Dysphagia did not occur. Dryness of the eyes was not reported.

### Example 15: Prevention of Radiation Damage by the Use of Botulinum Toxin Type B in the Therapeutic Use of ¹⁷⁷Lu-PSMA-617

Two weeks (13 days) prior to the therapeutic use of ¹⁷⁷Lu-PSMA-617, a total of 6,000 units of botulinum toxin type B (Neurobloc®) are applied. The botulinum toxin type B was applied asymmetrically (4,000 units) to the right parotid gland and to the contralateral, left submandibular gland (2,000 units).

Immediately prior to the first ¹⁷⁷Lu-PSMA-617 therapy/day 0, saliva production was normal. After two and a half weeks (day 0), the first administration of ¹⁷⁷Lu-PSMA-617 (6GBq/kg) took place to start the treatment of a prostate carcinoma patient.

After about eight weeks (day 56), another administration of ¹⁷⁷Lu-PSMA-617 (6 GBq/kg) was made. After completion of the treatment (two therapy cycles on days 0 and 56) and a follow-up period of approx. eight weeks, on day 116 normal salivary production remained unchanged. Dysphagia did not occur. No dryness of the eyes was reported.

Based on Examples 13-15, the following further treatments of the following four patient groups are planned:
1) Middle-aged patients 50-75 years
2) Older patients>75 years
3) Multimorbid patients and/or patients with mild cognitive impairment (MCI)
4) Patients with an expected short survival time<1.5 years

### Example 16: Planned Treatment of Patients of Patient Group 1 (Middle-Aged, 50-75 Years): Prevention of Radiation Damage from the Use of Botulinum Toxin Type A and an Anticholinergic in the Therapeutic Use of ²²⁵Ac-PSMA-617

Three weeks (21 days) prior to the therapeutic use of ²²⁵Ac-PSMA-617, a total of 150 units of botulinum toxin type A (Xeomin®) are applied. The botulinum toxin type A is applied on both sides (50 units each) to the parotid glands or the submandibular glands (25 units each) (i.e., in a ratio of 2/3 parotid gland to 1/3 submandibular gland).

The anticholinergic is additionally administered three days prior to therapy using ²²⁵ Ac-PSMA-617. The anticholinergic, here scopolamine, is applied in the form of a transdermal patch every three days. In this case, about 1 mg of scopolamine is released into the systemic circulation at an almost constant rate within 72 hours. Said scopolamine patch is replaced on day 0 and day 3 and day 6 of the ²²⁵Ac-PSMA-617 therapy, such that the patient is treated with additional scopolamine up to nine days after therapy. This approach also protects the sublingual salivary gland, the minor salivary glands, and the ocular glands

### Example 17: Planned Treatment of Patients of Patient Group 2 (Aged 75 Years or Older): Prevention of Radiation Damage from the Use of Botulinum Toxin Type B in the Therapeutic Use of ²²⁵Ac-PSMA-617

Two weeks (14 days) prior to the therapeutic use of ²²⁵Ac-PSMA-617, a total of 6,000 units of botulinum toxin type B (Neurobloc®) are applied. The botulinum toxin type B is applied to the parotid glands (2,000 units each) or the submandibular glands (1,000 units each) on both sides (i.e., in a ratio of 2/3 parotid gland to 1/3 mandibular salivary gland).

### Example 18: Planned Treatment of Patients in Patient Groups 3+4 (Multimorbid Patients/Patients with MCI/Patients with Reduced Life Expectancy): Prevention of Radiation Damage from the Use of Botulinum Toxin Type A in the Therapeutic Use of ²²⁵Ac-PSMA-617

Three weeks (21 days) prior to the therapeutic use of ²²⁵Ac-PSMA-617, a total of 150 units of botulinum toxin type A (Xeomin®) will be applied. The botulinum toxin type A will be applied asymmetrically on one side as a high-dose monotherapy (100 units) to the right parotid gland or on one side to the left submandibular gland (50 units) (i.e., in a ratio of 2/3 parotid gland to 1/3 submandibular gland).

The term high-dose monotherapy with botulinum toxin A (Xeomin®) refers to the respective single dose in the two glands, which is in a previously unpublished dose range per gland, but is well tolerated, as can be seen from the patients already treated. The glandular high-dose monotherapy must never be applied to all four glands in this patient group in order to avoid dysphagia. Asymmetric monotherapy with botulinum toxin type A in only one contralateral parotid and submandibular gland also serves to avoid side effects associated with anticholinergics in patient group 3. The two initially unchanged uninjected glands with initial saliva production, shorten (until they are completely damaged by the second, third or fourth ²²⁵Ac-PSMA-617 treatment) the duration of dry mouth during the therapy period at the expense of complete destruction of two of the four salivary glands due to repeated ²²⁵Ac-PSMA-617 treatments, and is therefore particularly suitable for patient group 4 with an expected short survival time in terms of quality of life.

1. A composition comprising botulinum toxin in combination with an anticholinergic for preventing radiation damage to the glands, wherein the radiation damage is caused by radioligands.

2. The composition according to claim 1, wherein the radiation damage is caused by a PSMA radioligand, wherein the radiation damage is selected from the group consisting of ²²⁵Ac-PSMA, ⁶⁸Ga-PSMA, ¹⁸F-PSMA or ¹⁷⁷Lu-PSMA.

3. (canceled)

4. The composition according to claim 1, wherein the botulinum toxin is selected from the group consisting of botulinum toxin types A, B, C, D, E, F and G.

5. The composition according to claim 1, wherein the botulinum toxin comprises between 1 and 10,000 units.

6. The composition according to claim 1, wherein the botulinum toxin is a botulinum toxin type A, preferably comprising between 1 and 1,500 units of botulinum toxin type A.

7. (canceled)

8. The composition according to claim 1, wherein the botulinum toxin is a botulinum toxin type B, preferably comprising between 100 and 10,000 units of botulinum toxin type B.

9. (canceled)

10. The botulinum toxin in combination with an anticholinergic for use according to any one of the preceding claims, wherein the botulinum toxin is a botulinum toxin type E or type F, preferably comprising between 1 and 10,000 units of botulinum toxin type E or type F.

11. (canceled)

12. The composition according to claim 1, wherein the anticholinergic is selected from the group consisting of tropicamide, atropine, scopolamine, glycopyrrolate, amitriptyline, clonidine, ipratropium bromide and trihexyphenidyl.

13. The composition according to claim 1, wherein the anticholinergic is to be administered transdermally, orally or intravenously.

14. The composition according to claim 1, wherein the anticholinergic is scopolamine, wherein the scopolamine is to be administered transdermally.

15. A botulinum toxin type B for the prevention of radiation damage to the parotid, submandibular, sublingual, minor salivary and ocular glands, wherein radiation damage is caused by radioligands.

16. A composition comprising a botulinum toxin type A for preventing radiation damage to the glands,
wherein the botulinum toxin type A is to be administered contralaterally into the respective parotid gland or mandibular gland or
wherein 100 units of botulinum toxin type A is to be administered into a parotid gland and 50 units of botulinum toxin type A is to be administered into the respective contralateral mandibular gland, or
wherein the botulinum toxin type A is to be administered in a dose ratio of 2/3 into the parotid glands and 1/3 into the submandibular glands.

17. (canceled)

18. (canceled)

19. The botulinum toxin type A composition according to claim 16, wherein the patient has a life expectancy of between six and twelve months.

20. The botulinum toxin type A composition according to claim 16, wherein the patient is aged 75 years or older.

21. The botulinum toxin type A composition according to claim 16, wherein the patient is suffering from either a mild cognitive impairment or dementia.

22. The botulinum toxin type A composition according to claim 16, wherein the patient is co- or multi-morbid.

23. The botulinum toxin type A composition according to claim 16 for lowering aquaporin-mediated radionuclide uptake in the submandibular and salivary gland cells.

24. The botulinum toxin type B composition according to claim 15,
wherein the botulinum toxin type B is to be administered in a dose ratio of 2/3 into the parotid glands and 1/3 into the submandibular glands or
wherein the botulinum toxin type B is to be administered contralaterally into the respective parotid gland or mandibular gland.

25. (canceled)

26. The composition according to claim 1,
wherein the botulinum toxin is to be administered one day to eight weeks prior to a diagnostic imaging procedure or prior to radiological treatment using one or more radioligands, or
wherein the anticholinergic is to be administered three days, two days, one day and on the same day prior to a diagnostic imaging procedure or prior to radiological treatment using one or more radionuclides, and about 7 to 30 days after the diagnostic imaging procedure or after a radiological treatment, or
wherein the botulinum toxin is to be administered one day to eight weeks and the anticholinergic is to be administered three days, two days, one day and on the same day prior to a diagnostic imaging procedure or prior to radiological treatment using one or more radionuclides, and about 7 to 30 days after the diagnostic imaging procedure or after a radiological treatment.

27. (canceled)

28. (canceled)

29. The composition according to claim 1, wherein the glands are exocrine and/or endocrine glands, preferably wherein the gland is a seromucous gland.

30. (canceled)

31. The composition according to claim 1, wherein the botulinum toxin A acts by down-regulating the α-1 adrenoceptors in the glands.

32. The composition according to claim 1, wherein the use is carried out in a group of patients, which has already undergone anti-androgen therapy or is currently undergoing anti-androgen therapy, while radioligands may cause radiation damage according to claim 1.
